(19) United States
(12) Patent Application Publication
(10) Pub. No.: US 2020/0155009 A1
(43) Pub. Date: May 21, 2020

(54) PHOTOACOUSTIC IMAGE GENERATION APPARATUS

(71) Applicant: FUJIFILM Corporation, Tokyo (JP)

(72) Inventor: Yukiya MIYACHI, Ashigarakami-gun (JP)

(73) Assignee: FUJIFILM Corporation, Tokyo (JP)

(21) Appl. No.: 16/750,962

(22) Filed: Jan. 23, 2020

Related U.S. Application Data
(63) Continuation of application No. PCT/JP2018/021753, filed on Jun. 6, 2018.

(30) Foreign Application Priority Data
Aug. 24, 2017 (JP) 2017-161242

(51) Int. Cl. A61B 5/00

(52) U.S. Cl. CPC A61B 5/0095; A61B 2576/00

(57) ABSTRACT

Provided is a photoacoustic image generation apparatus that can suppress degradation in image quality even in a case where some detections are inappropriately performed in a case where the detection is performed a plurality of times for acquiring detection signals for one photoacoustic image. In a case where a plurality of times of detections are required to acquire detection signals for one photoacoustic image, piezoelectric elements to be used and piezoelectric elements to be suspended are set alternately and continuously in an order of arrangement of the piezoelectric elements of a piezoelectric element array for the piezoelectric elements to be used for one time of detection, and the detection elements to be used are switched for each time of detection.

### CROSS-REFERENCE TO RELATED APPLICATIONS

This application is a Continuation of PCT International Application No. PCT/JP2018/021753 filed on Jun. 6, 2018, which claims priority under 35 U.S.C § 119(a) to Japanese Patent Application No. 2017-161242 filed on Aug. 24, 2017. Each of the above application(s) is hereby expressly incorporated by reference, in its entirety, into the present application.

### BACKGROUND OF THE INVENTION

### 1. Field of the Invention

The present invention relates to a photoacoustic image generation apparatus that generates a photoacoustic image by detecting a photoacoustic wave emitted from a tip portion of an insert inserted into a subject by a photoacoustic wave detection unit having a detection element array.

### 2. Description of the Related Art

An ultrasonography method has been known as a kind of image inspection method that can non-invasively inspect an internal state of a living body. An ultrasound probe that can transmit and receive ultrasonic waves is used in ultrasonography. In a case where the ultrasound probe transmits an ultrasonic wave to a subject (living body), the ultrasonic wave travels inside the living body and is reflected from an interface between tissues. The ultrasound probe receives the reflected ultrasonic wave and a distance is calculated based on a time until the reflected ultrasonic wave returns to the ultrasound probe. In this manner, it is possible to capture an image indicating the internal state of the living body.

In addition, photoacoustic imaging has been known which captures an image of the inside of a living body using a photoacoustic effect. In general, the inside of the living body is irradiated with pulsed laser light in the photoacoustic imaging. In the inside of the living body, the living body tissue absorbs energy of the pulsed laser light and an ultrasonic wave (photoacoustic wave) is generated by adiabatic expansion caused by the energy. The ultrasound probe or the like detects the photoacoustic wave and a photoacoustic image is formed based on the detection signal. In this manner, it is possible to visualize the inside of the living body based on the photoacoustic wave.

In addition, as a technique related to the photoacoustic imaging, JP2015-231583A discloses a puncture needle provided with a photoacoustic wave generation portion that absorbs light and generates a photoacoustic wave near a tip thereof. In the puncture needle, an optical fiber is provided up to the tip of the puncture needle and light guided by the optical fiber is emitted to the photoacoustic wave generation portion. An ultrasound probe detects the photoacoustic wave generated by the photoacoustic wave generation portion, and a photoacoustic image is generated based on the detection signal of the photoacoustic wave. A portion of the photoacoustic wave generation portion appears as a bright point in the photoacoustic image. Therefore, it is possible to check a position of the puncture needle using the photoacoustic image.

### SUMMARY OF THE INVENTION

Here, for example, in a case where the photoacoustic imaging is performed using the puncture needle as described in JP2015-231583A, the ultrasound probe for detecting the photoacoustic wave comprises a piezoelectric element array in which a plurality of piezoelectric elements are arranged.

The piezoelectric element array of the ultrasound probe is composed of, for example, 128 piezoelectric elements arranged one-dimensionally, and the detection signal detected by the piezoelectric elements is received by a receiving circuit through a multiplexer.

In general, there is a limit on the number of channels that can be simultaneously received by the receiving circuit as described in WO 2016/002258. For example, in a case where a 64-channel receiving circuit is used and the number of piezoelectric elements is 128 as described above, the number of channels of the receiving circuit is smaller than the number of piezoelectric elements in the piezoelectric element array. Therefore, the 128 detection elements are divided into detection element groups consisting of 64 detection elements, and each divided detection element group is selectively connected to the receiving circuit by a 1:2 multiplexer. In this case, detection is performed twice in order to generate one image. First detection is performed by any one of 64 piezoelectric elements in the right half of the 128 piezoelectric elements or 64 piezoelectric elements in the left half thereof, and second detection is performed by remaining half of the 64 piezoelectric elements thereof to generate the image using the detection signals of the 128 piezoelectric elements obtained by the two times of detections.

However, there is a case where one time of detection is inappropriately performed during the first detection and second detection, such as in a case where the ultrasound probe is displaced or the ultrasound probe is separated from the subject. In this case, the image quality may be degraded, such as extreme brightness differences appearing at a boundary of a detection region, and it may be difficult to specify a tip portion of the puncture needle.

In view of the above-described circumstances, an object of the invention is to provide a photoacoustic image generation apparatus that can suppress degradation in image quality even in a case where some detections are inappropriately performed, in a case where the detection is performed a plurality of times for acquiring detection signals for one photoacoustic image.

The photoacoustic image generation apparatus according to the embodiment of the invention comprises an insert of which at least a tip portion is inserted into a subject and which has a photoacoustic wave generation portion that absorbs light and generates a photoacoustic wave in the tip portion, a photoacoustic wave detection unit having a detection element array in which a plurality of detection elements that detect the photoacoustic wave and output a detection signal are arranged, a control unit that controls the photoacoustic wave detection unit to switch detection elements to be used in the detection element array for performing a plurality of times of detections in a case where detection signals for one photoacoustic image are acquired, and a photoacoustic image generation unit that generates a photoacoustic image based on the detection signals. The control unit sets detection elements to be used and detection elements to be suspended alternately and continuously in an order of arrangement of the detection elements of the detection element array for the detection elements to be used for one time of detection and switches the detection elements to be used for each time of detection.

Here, the “setting detection elements to be used and detection elements to be suspended alternately and continuously” means a state in which one detection element or a plurality of continuous detection elements to be used and one detection element or a plurality of continuous detection elements to be suspended are set alternately and continuously, and the detection elements to be used and the detection elements to be suspended are periodically disposed. The “setting detection elements to be used and detection elements to be suspended alternately and continuously” also includes a state in which the detection elements to be used and the detection elements to be suspended are not always required to be set alternately and continuously in a completely regular manner over the entire region of the detection element array, for example, a part of both end portions or the like of the detection element array is disposed irregularly.

In the photoacoustic image generation apparatus according to the embodiment of the invention, the control unit may control the photoacoustic wave detection unit to switch detection elements to be used in the detection element array for performing a times of detections of two or more, set b detection elements for continuous use and b×(a−1) detection elements for continuous suspension alternately and continuously in an order of arrangement of the detection elements of the detection element array for the detection elements to be used for one time of detection, and move the detection elements to be used on a b-by-b basis in the same direction for each time of detection, in a case where detection signals for one acoustic image are acquired. Hereinafter, the “moving the detection elements to be used” means that detection elements disposed at moved positions are used. For example, “moving the detection elements to be used by b detection elements” means that detection elements disposed adjacent to the used detection elements by b detection elements.

Here, “a” is an integer of 2 or more, and “b” is an integer of 1 or more.

In addition, the photoacoustic image generation unit may generate an acoustic image based on a remaining detection signal except for a detection signal for which a detection is not performed appropriately among the detection signals acquired by the plurality of times of detections, in a case where one acoustic image is generated.

In addition, the control unit may set one detection element for continuous use and one detection element for continuous suspension alternately and continuously in an order of arrangement of the detection elements of the detection element array for the detection elements to be used for one time of detection and move the detection elements to be used on a one-by-one basis in the same direction for each time of detection, in a case where two times of detections are performed for acquiring detection signals for one acoustic image.

In addition, the control unit may set one detection element for continuous use and two detection elements for continuous suspension alternately and continuously in an order of arrangement of the detection elements of the detection element array for the detection elements to be used for one time of detection and move the detection elements to be used on a one-by-one basis in the same direction for each time of detection, in a case where three times of detections are performed for acquiring detection signals for one acoustic image.

In addition, the control unit may set one detection element for continuous use and three detection elements for continuous suspension alternately and continuously in an order of arrangement of the detection elements of the detection element array for the detection elements to be used for one time of detection and move the detection elements to be used on a one-by-one basis in the same direction for each time of detection, in a case where four times of detections are performed for acquiring detection signals for one acoustic image.

In addition, the control unit may set two detection elements for continuous use and two detection elements for continuous suspension alternately and continuously in an order of arrangement of the detection elements of the detection element array for the detection elements to be used for one time of detection and move the detection elements to be used on a two-by-two basis in the same direction for each time of detection, in a case where two times of detections are performed for acquiring detection signals for one acoustic image.

The photoacoustic image generation apparatus according to the embodiment of the invention comprises an insert of which at least a tip portion is inserted into a subject and which has a photoacoustic wave generation portion that absorbs light and generates a photoacoustic wave in the tip portion, a photoacoustic wave detection unit having a detection element array in which a plurality of detection elements that detect the photoacoustic wave and output a detection signal are arranged, a control unit that controls the photoacoustic wave detection unit to switch detection elements to be used in the detection element array for performing a plurality of times of detections in a case where detection signals for one photoacoustic image are acquired, and a photoacoustic image generation unit that generates a photoacoustic image based on the detection signals. The control unit sets detection elements to be used and detection elements to be suspended alternately and continuously in an order of arrangement of the detection elements of the detection element array for the detection elements to be used for one time of detection and switches the detection elements to be used for each time of detection. Therefore, it is possible to suppress degradation in image quality even in a case where some detections are inappropriately performed.

### BRIEF DESCRIPTION OF THE DRAWINGS

FIG. 1 is a block diagram illustrating the schematic configuration of a photoacoustic image generation apparatus which is an embodiment of the invention.

FIG. 2 is a cross-sectional view of the configuration of a tip portion of a puncture needle.

FIG. 3 is a block diagram illustrating the schematic configuration of an acoustic wave detection unit.

FIG. 4 is a diagram illustrating a use state of piezoelectric elements in a piezoelectric element array at each time of detection.

FIG. 5 is a diagram illustrating a use state of piezoelectric elements in the piezoelectric element array at each time of detection.

FIG. 6 is a diagram illustrating an example of original data acquired in a case where detection is appropriately performed in the photoacoustic image generation apparatus according to the embodiment of the invention.

FIG. 7 is a diagram illustrating an image after phase matching addition is performed on the original data in FIG. 6.

FIG. 8 is a diagram illustrating an example of original data acquired in a case where detection is inappropriately performed in a photoacoustic image generation apparatus in the related art.

FIG. 9 is a diagram illustrating an image after the phase matching addition is performed on the original data in FIG. 8.

FIG. 10 is a diagram illustrating an example of original data acquired in a case where detection is inappropriately performed in the photoacoustic image generation apparatus according to the embodiment of the invention.

FIG. 11 is a diagram illustrating an image after the phase matching addition is performed on the original data in FIG. 10.

FIG. 12 is a diagram illustrating an image obtained by performing nearest neighbor interpolation on the original data in FIG. 10.

FIG. 13 is a diagram illustrating an image obtained by performing linear interpolation on the original data in FIG. 10.

### DESCRIPTION OF THE PREFERRED EMBODIMENTS

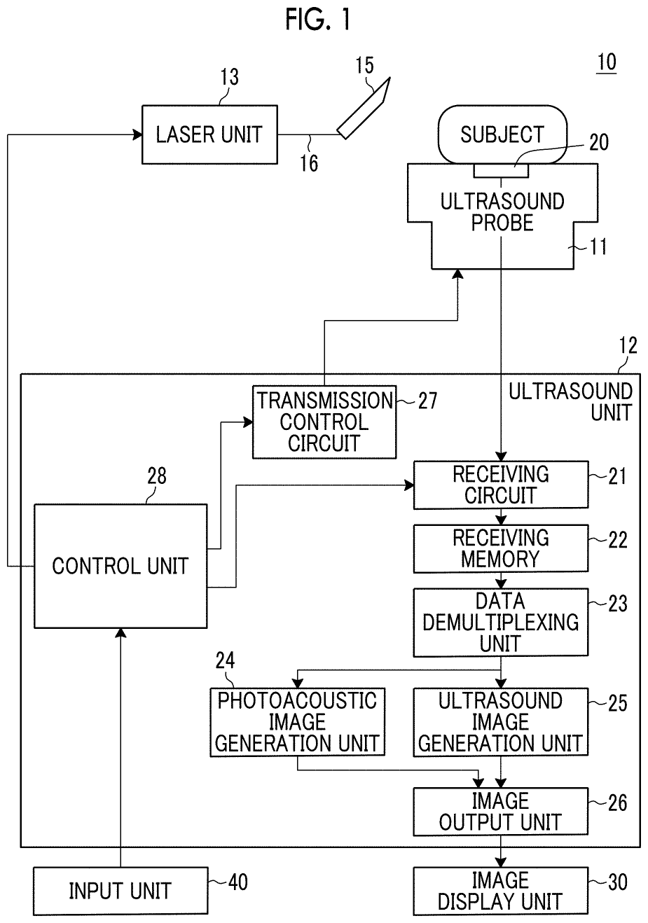

Hereinafter, a photoacoustic image generation apparatus according to an embodiment of the invention will be described in detail with reference to drawings. FIG. 1 is a diagram illustrating a schematic configuration of a photoacoustic image generation apparatus 10 according to the embodiment.

As illustrated in FIG. 1, the photoacoustic image generation apparatus 10 according to the embodiment comprises an ultrasound probe 11, an ultrasound unit 12, a laser unit 13, and a puncture needle 15 (corresponding to an insert according to the embodiment of the invention). The puncture needle 15 and the laser unit 13 are connected by an optical cable 16 having an optical fiber. The puncture needle 15 can be attached to and detached from the optical cable 16 and is disposable. An ultrasonic wave is used as an acoustic wave in the embodiment, but the invention is not limited to the ultrasonic wave. An acoustic wave with an audible frequency may be used as long as an appropriate frequency is selected according to, for example, an inspection target or a measurement condition.

The laser unit 13 comprises a solid-state laser light source using, for example, yttrium aluminum garnet (YAG) or alexandrite. Laser light emitted from the solid-state laser light source of the laser unit 13 is guided by the optical cable 16 and is incident on the puncture needle 15. The laser unit 13 according to the embodiment emits pulsed laser light in a near-infrared wavelength range. The near-infrared wavelength range means a wavelength range of 700 nm (nanometer) to 850 nm (nanometer). The solid-state laser light source is used in the embodiment. However, another laser light source such as a gas laser light source may be used or a light source other than the laser light source may be used.

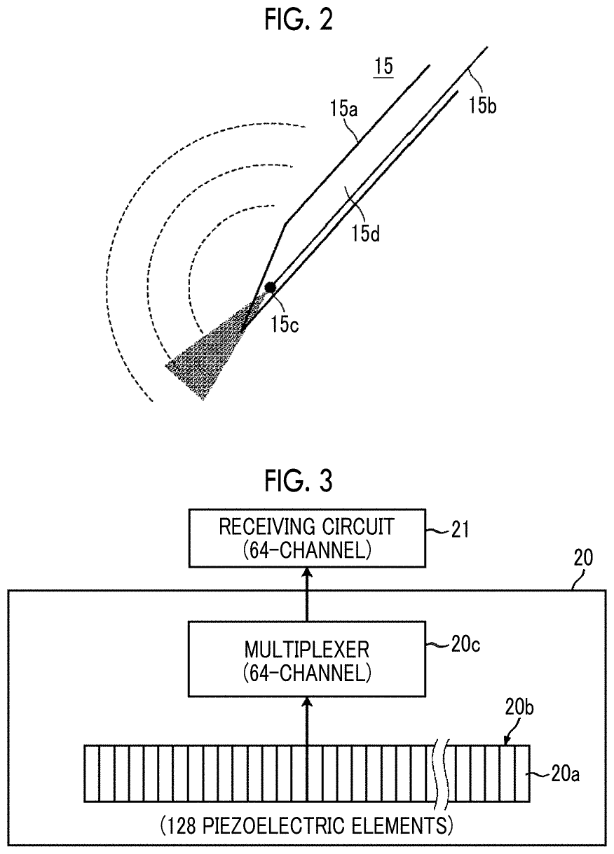

The puncture needle 15 is an embodiment of the insert according to the invention and is a needle that is inserted into a subject. FIG. 2 is a cross-sectional view including a center axis that extends in a length direction of the puncture needle 15. The puncture needle 15 includes a puncture needle main body 15a that has an opening at an acute tip and is formed in a hollow shape, an optical fiber 15b (corresponding to a light guide member according to the embodiment of the invention) that guides laser light emitted from the laser unit 13 to the vicinity of the opening of the puncture needle 15, and a photoacoustic wave generation portion (a photoacoustic wave generation member) 15c that absorbs laser light emitted from the optical fiber 15b and generates a photoacoustic wave.

The optical fiber 15b and the photoacoustic wave generation portion 15c are disposed in a hollow portion 15d of the puncture needle main body 15a. For example, the optical fiber 15b is connected to the optical fiber in the optical cable 16 (refer to FIG. 1) through an optical connector provided at a base end of the puncture needle 15. For example, laser light of 0.2 mJ (millijoule) is emitted from a light emission end of the optical fiber 15b.

The photoacoustic wave generation portion 15c is provided at the light emission end of the optical fiber 15b and is provided near the tip of the puncture needle 15 and in the inner wall of the puncture needle main body 15a. The photoacoustic wave generation portion 15c absorbs the laser light emitted from the optical fiber 15b and generates the photoacoustic wave. The photoacoustic wave generation portion 15c is made of, for example, an epoxy resin, a polyurethane resin, a fluorine resin, and silicone rubber with which a black pigment is mixed. The photoacoustic wave generation portion 15c is illustrated to be larger than the optical fiber 15b in FIG. 2, but the invention is not limited thereto. The photoacoustic wave generation portion 15c may have a size that is equal to a diameter of the optical fiber 15b.

The photoacoustic wave generation portion 15c is not limited to the above, and a metal film or an oxide film having light absorptivity with respect to the wavelength of laser light may be used as the photoacoustic wave generation portion. An oxide film made of, for example, iron oxide, chromium oxide, or manganese oxide having high light absorptivity with respect to the wavelength of laser light can be used as the photoacoustic wave generation portion 15c. Alternatively, a metal film made of, for example, titanium (Ti) or platinum (Pt) that has lower light absorptivity than an oxide but higher biocompatibility than an oxide may be used as the photoacoustic wave generation portion 15c. In addition, a position where the photoacoustic wave generation portion 15c is provided is not limited to the inner wall of the puncture needle main body 15a. For example, a metal film or an oxide film which is the photoacoustic wave generation portion 15c may be formed on the light emission end of the optical fiber 15b with a film thickness of about 100 nm (nanometer) by vapor deposition or the like such that the metal film or the oxide film covers the light emission end. In this case, at least a part of the laser light emitted from the light emission end of the optical fiber 15b is absorbed by the metal film or the oxide film covering the light emission end and the photoacoustic wave is generated from the metal film or the oxide film.

Returning to FIG. 1, the ultrasound probe 11 detects the photoacoustic wave emitted from the photoacoustic wave generation portion 15c after the puncture needle 15 is inserted into the subject. The ultrasound probe 11 comprises an acoustic wave detection unit 20 (corresponding to the photoacoustic wave detection unit according to the embodiment of the invention) that detects a photoacoustic wave.

As shown in FIG. 3, the acoustic wave detection unit 20 comprises a piezoelectric element array 20b (corresponding to the detection element array according to the embodiment of the invention) in which 128 piezoelectric elements 20a (corresponding to the detection element according to the embodiment of the invention) for detecting the photoacoustic wave are arranged one-dimensionally, and a multiplexer 20c. The piezoelectric element 20a is an ultrasound transducer and is made of, for example, piezoelectric ceramics or a polymer film such as polyvinylidene fluoride (PVDF). In addition, the acoustic wave detection unit 20 comprises an acoustic lens, an acoustic matching layer, a backing member, a control circuit of the piezoelectric element array 20b, and the like (not illustrated).

The number and arrangement of the piezoelectric elements 20a are not limited thereto. For example, the number of the piezoelectric elements 20a may be 192 or 256, or may be two-dimensionally arranged. In this specification, an arrangement direction of the piezoelectric elements may be an one-dimensional direction in a case where the piezoelectric elements are arranged one-dimensionally, or may be any direction of two-dimensional directions perpendicular to each other in a case where the piezoelectric elements are arranged two-dimensionally. It is preferable to set a direction in which the arranged piezoelectric elements are large as the arrangement direction.

The multiplexer 20c selectively connects piezoelectric elements to be used for one time of detection among the piezoelectric elements 20a constituting the piezoelectric element array 20b to the receiving circuit 21 of the ultrasound unit 12.

With the selective connection by the multiplexer 20c described above, a reception region of the photoacoustic wave of the piezoelectric element array 20b is divided into a plurality of reception regions and a detection signal of the photoacoustic wave for each reception region is acquired.

Specifically, the multiplexer 20c according to the embodiment has 64 channels and can simultaneously acquire detection signals of 64 piezoelectric elements. The 64 piezoelectric elements connected to the receiving circuit 21 by the multiplexer 20c are specified by a control unit 28 of the ultrasound unit 12. The control unit 28 switches the reception region of the photoacoustic wave by switching the 64 piezoelectric elements to acquire a detection signal of the entire reception region of the piezoelectric element array 20b.

With the piezoelectric element array 20b of the acoustic wave detection unit 20, the ultrasound probe 11 transmits the acoustic wave (ultrasonic wave) to the subject and receives the reflected acoustic wave (reflected ultrasonic wave) with respect to the transmitted ultrasonic wave, in addition to the detection of the photoacoustic wave. The transmission and reception of the ultrasonic wave may be performed at separated positions. For example, the ultrasonic wave may be transmitted from a position different from the ultrasound probe 11, and the piezoelectric element array 20b of the ultrasound probe 11 may receive the reflected ultrasonic wave with respect to the transmitted ultrasonic wave. It is possible to use a linear ultrasound probe, a convex ultrasound probe, a sector ultrasound probe, or the like as the ultrasound probe 11.

The ultrasound unit 12 has the receiving circuit 21, a receiving memory 22, a data demultiplexing unit 23, a photoacoustic image generation unit 24, an ultrasound image generation unit 25, an image output unit 26, a transmission control circuit 27, and the control unit 28. The ultrasound unit 12 typically has, for example, a processor, a memory, and a bus. In the ultrasound unit 12, a program relating to photoacoustic image generation processing, ultrasound image generation processing, and reception region switching processing of the acoustic wave of the piezoelectric element array 20b in the acoustic wave detection unit 20 is incorporated in the memory. The program is operated by the control unit 28 which is formed by the processor to realize functions of the data demultiplexing unit 23, the photoacoustic image generation unit 24, the ultrasound image generation unit 25, the image output unit 26. That is, each of these units is formed by the memory into which the program is incorporated and the processor.

The hardware configuration of the ultrasound unit 12 is not particularly limited and can be realized by combining a plurality of integrated circuits (ICs), a processor, an application specific integrated circuit (ASIC), a field-programmable gate array (FPGA), a memory, and the like as appropriate.

The receiving circuit 21 receives a detection signal output from the ultrasound probe 11 and stores the received detection signal in the receiving memory 22. The receiving circuit 21 of the embodiment has 64 channels. The receiving circuit 21 typically includes a low-noise amplifier, a variable-gain amplifier, a low pass filter, and an analog to digital converter (AD). The detection signal of the ultrasound probe 11 is amplified by the low-noise amplifier, is subjected to gain adjustment corresponding to a depth by the variable-gain amplifier, is converted into a digital signal by the AD converter after high-frequency components of the detection signal is cut by the low pass filter, and then is stored in the receiving memory 22. The receiving circuit 21 is formed by, for example, one IC.

The ultrasound probe 11 outputs a detection signal of the photoacoustic wave and a detection signal of the reflected ultrasonic wave. The receiving memory 22 stores the AD-converted detection signals (sampling data) of the photoacoustic wave and the reflected ultrasonic wave. The data demultiplexing unit 23 reads out the detection signal of the photoacoustic wave from the receiving memory 22 and transmits the detection signal to the photoacoustic image generation unit 24. The data demultiplexing unit 23 reads out the detection signal of the reflected ultrasonic wave from the receiving memory 22 and transmits the detection signal to the ultrasound image generation unit 25.

The photoacoustic image generation unit 24 generates a photoacoustic image based on the detection signal of the photoacoustic wave detected by the ultrasound probe 11. The photoacoustic image generation processing includes, for example, image reconfiguration such as phase matching addition, detection, and logarithmic conversion. The ultrasound image generation unit 25 generates an ultrasound image (reflected acoustic wave image) based on the detection signal of the reflected ultrasonic wave detected by the ultrasound probe 11. The ultrasound image generation processing also includes image reconfiguration such as phase matching addition, detection, logarithmic conversion, and the like. The image output unit 26 outputs the photoacoustic image and the ultrasound image on an image display unit 30 such as a display apparatus.

The control unit 28 controls each unit in the ultrasound unit 12. In a case where a photoacoustic image is acquired, the control unit 28 transmits a trigger signal to the laser unit 13 to cause the laser unit 13 to emit laser light. In addition, the control unit 28 transmits a sampling trigger signal to the receiving circuit 21 to control a sampling start timing of the photoacoustic wave or the like with the emission of the laser light.

The image display unit 30 may display the photoacoustic wave image and the ultrasound image separately or combinedly. Since it becomes possible to check where the tip of the puncture needle 15 is in the living body with the combined display, thus accurate and safe puncture is possible.

Here, the control unit 28 according to the embodiment controls the multiplexer 20c of the acoustic wave detection unit 20 as described above to set the 64 piezoelectric elements to be connected to the receiving circuit 21 for one time of detection. In the ultrasound probe 11 according to the embodiment, a 64-channel receiving circuit 21 is combined with the piezoelectric element array 20b consisting of 128 piezoelectric elements 20a. Therefore, two times of detections (128÷64=2) are required to acquire detection signals for one acoustic image.

The control unit 28 sets piezoelectric elements 20a to be used and piezoelectric elements 20a to be suspended alternately and continuously in the order of arrangement of the piezoelectric elements 20a of the piezoelectric element array 20b for the piezoelectric elements 20a to be used for one time of detection and switches the piezoelectric elements 20a to be used for each time of detection. As an example in the embodiment, one piezoelectric element for use and one piezoelectric element for suspension are set alternately and continuously in the order of arrangement of the piezoelectric elements 20a of the piezoelectric element array 20b, and the piezoelectric elements 20a to be used are moved to adjacent piezoelectric elements 20a by one piezoelectric element 20a for each time of detection. That is, piezoelectric elements 20a disposed adjacent to the used piezoelectric elements 20a by one piezoelectric element 20a is used in the next detection.

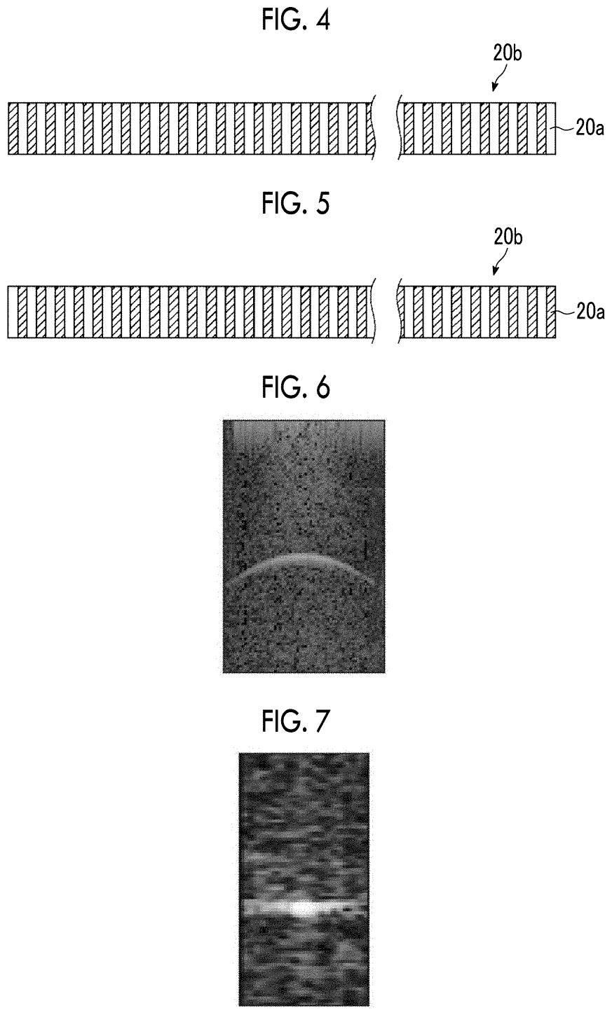

FIGS. 4 and 5 are diagrams illustrating use states of the piezoelectric element array 20b, and hatched piezoelectric elements 20a in the figures indicate the piezoelectric elements 20a to be used.

Specifically, in the first detection as shown in FIG. 4, one piezoelectric element for use and one piezoelectric element for suspension are set alternately and continuously such as use, suspension, use, suspension, and so on from the left in order for the piezoelectric elements 20a to be used. In the second detection as shown in FIG. 5, the piezoelectric elements 20a to be used are moved to adjacent piezoelectric elements 20a by one piezoelectric element 20a from the first detection, and one piezoelectric element for use and one piezoelectric element for suspension are set alternately and continuously such as suspension, use, suspension, use, and so on from the left in order for the piezoelectric elements 20a to be used.

The control unit 28 performs the detection twice as described above to acquire the detection signal of the photoacoustic wave in the entire reception region of the piezoelectric element array 20b. In a case where the photoacoustic image is acquired, the control unit 28 performs control which generates the photoacoustic wave for each time of detection.

The data demultiplexing unit 23 transmits the detection signal of the entire reception region from the receiving memory 22 to the photoacoustic image generation unit 24 and the photoacoustic image generation unit 24 generates a photoacoustic image of one frame, or the data demultiplexing unit 23 transmits the detection signal of the entire reception region from the receiving memory 22 to the ultrasound image generation unit 25 and the ultrasound image generation unit 25 generates an ultrasound image of one frame.

Here, a case where a photoacoustic image is generated in the photoacoustic image generation apparatus 10 according to the embodiment will be described as an example. FIG. 6 is an example of an image of original data (image before phase matching addition) in a case where a photoacoustic wave generated from the tip of the puncture needle 15 is normally received using the piezoelectric element array 20b consisting of 128 piezoelectric elements 20a. This figure shows a state where the tip of the puncture needle 15 is at a position facing the vicinity of the center of the piezoelectric element array 20b. In a case where the photoacoustic image generation unit 24 performs the phase matching addition on the original data shown in FIG. 6, it is possible to check a bright point indicating the tip of the puncture needle 15 near the center in a shape close to a circle as shown in FIG. 7.

However, there is a case where one time of detection is inappropriately performed during the first detection and second detection, such as in a case where the ultrasound probe 11 is displaced or the ultrasound probe 11 is separated from the subject. In this case, image quality may be degraded, such as extreme brightness differences appearing at a boundary of a detection region.

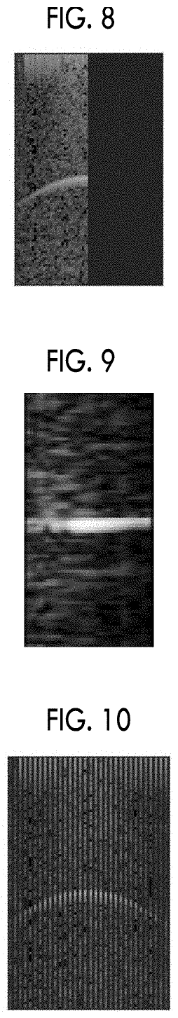

FIG. 8 is an example of an image of original data in a case where a photoacoustic wave generated from the tip of the puncture needle is received using a piezoelectric element array consisting of 128 piezoelectric elements similar to in FIG. 6. This figure shows a case where the second detection is inappropriately performed (case where no signal is received) in a method in the related art in which the left half of the piezoelectric element array is used for the first detection and the right half thereof is used for the second detection. In a case where the photoacoustic image generation unit 24 performs the phase matching addition on the original data shown in FIG. 8, the resolution of the right half is degraded and a bright point indicating the tip of the puncture needle becomes unclear near the center as shown in FIG. 9.

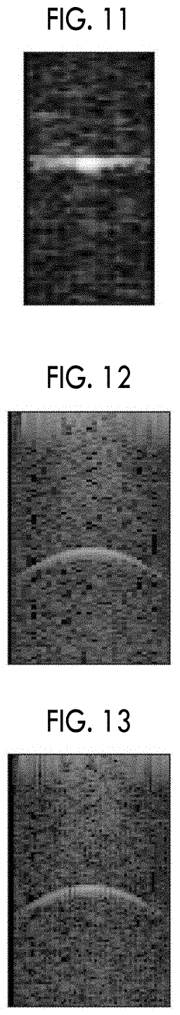

FIG. 10 is an example of an image of original data in a case where a photoacoustic wave generated from the tip of the puncture needle 15 is normally received using the piezoelectric element array 20b consisting of 128 piezoelectric elements 20a, similar to in FIG. 6. This figure shows a case where the second detection is inappropriately performed (case where no signal is received) in the method according to the embodiment in which each piezoelectric element 20a is alternately used on a one-by-one basis, such as use, suspension, use, suspension, and so on from the left in order for the piezoelectric elements 20a to be used for the first detection as shown in FIG. 4 and each piezoelectric element 20a is alternately used on a one-by-one basis, such as suspension, use, suspension, use, and so on from the left in order for the piezoelectric elements 20a to be used for the second detection as shown in FIG. 5. In a case where the photoacoustic image generation unit 24 performs the phase matching addition on the original data shown in FIG. 10, it is possible to check a bright point indicating the tip of the puncture needle 15 near the center in a shape close to a circle as shown in FIG. 11 as compared with the abnormal example in the method in the related art shown in FIG. 9 although not as much as the normal example shown in FIG. 7.

For the photoacoustic image generation unit 24 and the ultrasound image generation unit 25 according to the embodiment of the invention, an acoustic image may be generated based on a remaining detection signal except for a detection signal for which a detection is not performed appropriately among the detection signals acquired by the plurality of times (twice in the embodiment) of detections in a case where one acoustic image is generated.

For example, detection signal intensity data (total value or average value) of each time of detection may be compared for determination of whether or not the detection is appropriately performed in each time of detection. Specifically, in a case where a difference between the detection signal intensity data is less than a predetermined value (for example, 1.5 times), the image is generated using all the detection signals acquired by the two times of detections. In a case where the difference between the detection signal intensity data is equal to or larger than the predetermined value (for example, 1.5 times), the image is generated using only detection signal (for example, larger detection signal intensity data) acquired by any one of the detection signals acquired by the two times of detections. Interpolation is performed on a region of a detection signal that is not used in this case based on the used detection signal.

FIG. 12 is an example in which nearest neighbor interpolation is performed in the example of FIG. 10, and FIG. 13 is an example in which linear interpolation is performed in the example of FIG. 10. The linear interpolation can reproduce a delay curve at the tip of the puncture needle 15 more finely than the nearest neighbor interpolation, but tends to generate noise. Any interpolation may be used since a difference in the influence on the final image quality is slight. The interpolation processing is not limited to the nearest neighbor interpolation or the linear interpolation, and any known method may be used.

As described above, in a case where the detection signals for one acoustic image are acquired in two times of detections, the photoacoustic image generation apparatus 10 according to the embodiment can suppress the degradation in image quality even in a case where any one of the detections is inappropriately performed, since each piezoelectric element 20a is alternately used on a one-by-one basis such as use, suspension, use, suspension, and so on and the piezoelectric elements 20a to be used in each time of detection are distributed to the entire piezoelectric element array 20b.

The number of piezoelectric elements 20a in the piezoelectric element array 20b, the number of channels in the receiving circuit 21, and the arrangement of the piezoelectric elements 20a to be used in each time of detection in the case where the plurality of times of detections are performed for acquiring the detection signals for one acoustic image are not limited to the above, but various forms can be employed.

For example, in a case where a 64-channel receiving circuit 21 is combined with a piezoelectric element array 20b consisting of 192 piezoelectric elements 20a, three times of detections (192÷64=3) are required to acquire detection signals for one acoustic image.

The control unit 28 sets one piezoelectric element for use and two piezoelectric elements for suspension alternately and continuously in the order of arrangement of the piezoelectric elements 20a of the piezoelectric element array 20b for the piezoelectric elements 20a to be used for one time of detection and moves the piezoelectric elements 20a to be used on a one-by-one basis in the same direction for each time of detection.

For example, in a first detection, one piezoelectric element for use and two piezoelectric elements for suspension are set alternately and continuously such as use, suspension, suspension, use, suspension, suspension, and so on from the left in order for the piezoelectric elements 20a to be used. In a second detection, the piezoelectric elements 20a to be used are moved to a right side by one piezoelectric element 20a from the first detection, and one piezoelectric element for use and two piezoelectric elements for suspension are set alternately and continuously such as suspension, use, suspension, suspension, use, suspension, and so on from the left in order for the piezoelectric elements 20a to be used. In a third detection, the piezoelectric elements 20a to be used are moved to the right side by one piezoelectric element 20a from the second detection, and one piezoelectric element for use and two piezoelectric elements for suspension are set alternately and continuously such as suspension, suspension, use, suspension, suspension, use, and so on from the left in order for the piezoelectric elements 20a to be used.

In another example, in a case where a 64-channel receiving circuit 21 is combined with a piezoelectric element array 20b consisting of 256 piezoelectric elements 20a, four times of detections (256÷64=4) are required to acquire detection signals for one acoustic image.

The control unit 28 sets one piezoelectric element for use and three piezoelectric elements for suspension alternately and continuously in the order of arrangement of the piezoelectric elements 20a of the piezoelectric element array 20b for the piezoelectric elements 20a to be used for one time of detection and moves the piezoelectric elements 20a to be used on a one-by-one basis in the same direction for each time of detection.

For example, in a first detection, one piezoelectric element for use and three piezoelectric elements for suspension are set alternately and continuously such as use, suspension, suspension, suspension, use, suspension, suspension, suspension, and so on from the left in order for the piezoelectric elements 20a to be used. In a second detection, the piezoelectric elements 20a to be used are moved to a right side by one piezoelectric element 20a from the first detection, and one piezoelectric element for use and three piezoelectric elements for suspension are set alternately and continuously such as suspension, use, suspension, suspension, suspension, use, suspension, suspension, and so on from the left in order for the piezoelectric elements 20a to be used. In a third detection, the piezoelectric elements 20a to be used are moved to the right side by one piezoelectric element 20a from the second detection, and one piezoelectric element for use and three piezoelectric elements for suspension are set alternately and continuously such as suspension, suspension, use, suspension, suspension, suspension, use, suspension, and so on from the left in order for the piezoelectric elements 20a to be used. In a fourth detection, the piezoelectric elements 20a to be used are moved to the right side by one piezoelectric element 20a from the third detection, and one piezoelectric element for use and three piezoelectric elements for suspension are set alternately and continuously such as suspension, suspension, suspension, use, suspension, suspension, suspension, use, and so on from the left in order for the piezoelectric elements 20a to be used.

Furthermore, in another example, in a case where a 64-channel receiving circuit 21 is combined with the piezoelectric element array 20b consisting of 128 piezoelectric elements 20a, two times of detections (128÷64=2) are required to acquire detection signals for one acoustic image.

The control unit 28 sets two piezoelectric elements for use and two piezoelectric elements for suspension alternately and continuously in the order of arrangement of the piezoelectric elements 20a of the piezoelectric element array 20b for the piezoelectric elements 20a to be used for one time of detection and moves the piezoelectric elements 20a to be used on a two-by-two basis in the same direction for each time of detection.

For example, in a first detection, two piezoelectric elements for use and two piezoelectric elements for suspension are set alternately and continuously such as use, use, suspension, suspension, use, use, suspension, suspension, and so on from the left in order for the piezoelectric elements 20a to be used. In a second detection, the piezoelectric elements 20a to be used are moved to a right side by two piezoelectric elements 20a from the first detection, and two piezoelectric elements for use and two piezoelectric elements for suspension are set alternately and continuously such as suspension, suspension, use, use, suspension, suspension, use, use, and so on from the left in order for the piezoelectric elements 20a to be used.

In addition to the above, in a case where a times of detections are required to acquire detection signals for one acoustic image, b piezoelectric elements for continuous use and b×(a−1) piezoelectric elements for continuous suspension may be set alternately and continuously in the order of arrangement of the piezoelectric elements 20a of the piezoelectric element array 20b for the piezoelectric elements 20a to be used for one time of detection, and the piezoelectric elements 20a to be used may be moved on a b-by-b basis in the same direction for each time of detection. Furthermore, piezoelectric elements 20a to be used and piezoelectric elements 20a to be suspended may be set alternately and continuously in the order of arrangement of the piezoelectric elements 20a of the piezoelectric element array 20b for the piezoelectric elements 20a to be used for one time of detection, and the piezoelectric elements 20a to be used may be switched for each time of detection.

In the above embodiments, the puncture needle 15 is used as an embodiment of the insert. However, the insert is not limited thereto. The insert may be a radio-frequency ablation needle including an electrode that is used for radio-frequency ablation therein, a catheter that is inserted into a blood vessel, or a guide wire for a catheter that is inserted into a blood vessel. Alternatively, the insert may be an optical fiber for laser treatment.

The insert according to the embodiment of the invention is not limited to a needle, such as an injection needle, and may be a biopsy needle used for biopsy. That is, the needle may be a biopsy needle that is inserted into an inspection target of the living body and extracts the tissues of a biopsy site of the inspection target. In this case, a photoacoustic wave may be generated from an extraction portion (intake port) for sucking and extracting the tissues of the biopsy site. In addition, the needle may be used as a guiding needle that is used for insertion into a deep portion, such as a portion under the skin or an organ inside the abdomen.

The invention has been described above based on the preferred embodiments.

However, the insert and the photoacoustic image generation apparatus according to the embodiment of the invention are not limited only to the above embodiments. Various modifications and changes of the configurations according to the above embodiments are also included in the scope of the invention.

### EXPLANATION OF REFERENCES

10: photoacoustic image generation apparatus
11: ultrasound probe
12: ultrasound unit
13: laser unit
15: puncture needle
15a: puncture needle main body
15b: optical fiber
15c: photoacoustic wave generation portion
15d: hollow portion
16: optical cable
20: acoustic wave detection unit
20a: piezoelectric element
20b: piezoelectric element array
20c: multiplexer
21: receiving circuit
22: receiving memory
23: data demultiplexing unit
24: photoacoustic image generation unit
25: ultrasound image generation unit
26: image output unit
27: transmission control circuit
28: control unit
30: image display unit
40: input unit

What is claimed is:

1. A photoacoustic image generation apparatus comprising:
an insert of which at least a tip portion is inserted into a subject and which has a photoacoustic wave generation portion that absorbs light and generates a photoacoustic wave in the tip portion;
a probe having a detection element array in which a plurality of detection elements that detect the photoacoustic wave and output a detection signal are arranged; and
a processor,
wherein the processor is configured to
control the probe to switch detection elements to be used in the detection element array for performing a plurality of times of detections in a case where detection signals for one photoacoustic image are acquired,
generate a photoacoustic image based on the detection signals, and
set detection elements to be used and detection elements to be suspended alternately and continuously in an order of arrangement of the detection elements of the detection element array for the detection elements to be used for one time of detection and switch the detection elements to be used for each time of detection.

2. The photoacoustic image generation apparatus according to claim 1,
wherein the processor is further configured to control the probe to switch detection elements to be used in the detection element array for performing a times of detections of two or more, set b detection elements for continuous use and b×(a−1) detection elements for continuous suspension alternately and continuously in an order of arrangement of the detection elements of the detection element array for the detection elements to be used for one time of detection, and move the detection elements to be used on a b-by-b basis in the same direction for each time of detection, in a case where detection signals for one acoustic image are acquired.

3. The photoacoustic image generation apparatus according to claim 1,
wherein the processor is further configured to generate an acoustic image based on a remaining detection signal except for a detection signal for which a detection is not performed appropriately among the detection signals acquired by the plurality of times of detections, in a case where one acoustic image is generated.

4. The photoacoustic image generation apparatus according to claim 2,
wherein the processor is further configured to generate an acoustic image based on a remaining detection signal except for a detection signal for which a detection is not performed appropriately among the detection signals acquired by the plurality of times of detections, in a case where one acoustic image is generated.

5. The photoacoustic image generation apparatus according to claim 1,
wherein the processor is further configured to set one detection element for continuous use and one detection element for continuous suspension alternately and continuously in an order of arrangement of the detection elements of the detection element array for the detection elements to be used for one time of detection and move the detection elements to be used on a one-by-one basis in the same direction for each time of detection, in a case where two times of detections are performed for acquiring detection signals for one acoustic image.

6. The photoacoustic image generation apparatus according to claim 2,
wherein the processor is further configured to set one detection element for continuous use and one detection element for continuous suspension alternately and continuously in an order of arrangement of the detection elements of the detection element array for the detection elements to be used for one time of detection and move the detection elements to be used on a one-by-one basis in the same direction for each time of detection, in a case where two times of detections are performed for acquiring detection signals for one acoustic image.

7. The photoacoustic image generation apparatus according to claim 3,
wherein the processor is further configured to set one detection element for continuous use and one detection element for continuous suspension alternately and continuously in an order of arrangement of the detection elements of the detection element array for the detection elements to be used for one time of detection and move the detection elements to be used on a one-by-one basis in the same direction for each time of detection, in a case where two times of detections are performed for acquiring detection signals for one acoustic image.

8. The photoacoustic image generation apparatus according to claim 4,
wherein the processor is further configured to set one detection element for continuous use and one detection element for continuous suspension alternately and continuously in an order of arrangement of the detection elements of the detection element array for the detection elements to be used for one time of detection and move the detection elements to be used on a one-by-one basis in the same direction for each time of detection, in a case where two times of detections are performed for acquiring detection signals for one acoustic image.

9. The photoacoustic image generation apparatus according to claim 1,
wherein the processor is further configured to set one detection element for continuous use and two detection elements for continuous suspension alternately and continuously in an order of arrangement of the detection elements of the detection element array for the detection elements to be used for one time of detection and move the detection elements to be used on a one-by-one basis in the same direction for each time of detection, in a case where three times of detections are performed for acquiring detection signals for one acoustic image.

10. The photoacoustic image generation apparatus according to claim 2,
wherein the processor is further configured to set one detection element for continuous use and two detection elements for continuous suspension alternately and continuously in an order of arrangement of the detection elements of the detection element array for the detection elements to be used for one time of detection and move the detection elements to be used on a one-by-one basis in the same direction for each time of detection, in a case where three times of detections are performed for acquiring detection signals for one acoustic image.

11. The photoacoustic image generation apparatus according to claim 3,
wherein the processor is further configured to set one detection element for continuous use and two detection elements for continuous suspension alternately and continuously in an order of arrangement of the detection elements of the detection element array for the detection elements to be used for one time of detection and move the detection elements to be used on a one-by-one basis in the same direction for each time of detection, in a case where three times of detections are performed for acquiring detection signals for one acoustic image.

12. The photoacoustic image generation apparatus according to claim 4,
wherein the processor is further configured to set one detection element for continuous use and two detection elements for continuous suspension alternately and continuously in an order of arrangement of the detection elements of the detection element array for the detection elements to be used for one time of detection and move the detection elements to be used on a one-by-one basis in the same direction for each time of detection, in a case where three times of detections are performed for acquiring detection signals for one acoustic image.

13. The photoacoustic image generation apparatus according to claim 1,
wherein the processor is further configured to set one detection element for continuous use and three detection elements for continuous suspension alternately and continuously in an order of arrangement of the detection elements of the detection element array for the detection elements to be used for one time of detection and move the detection elements to be used on a one-by-one basis in the same direction for each time of detection, in a case where four times of detections are performed for acquiring detection signals for one acoustic image.

14. The photoacoustic image generation apparatus according to claim 2,
wherein the processor is further configured to set one detection element for continuous use and three detection elements for continuous suspension alternately and continuously in an order of arrangement of the detection elements of the detection element array for the detection elements to be used for one time of detection and move the detection elements to be used on a one-by-one basis in the same direction for each time of detection, in a case where four times of detections are performed for acquiring detection signals for one acoustic image.

15. The photoacoustic image generation apparatus according to claim 3,
wherein the processor is further configured to set one detection element for continuous use and three detection elements for continuous suspension alternately and continuously in an order of arrangement of the detection elements of the detection element array for the detection elements to be used for one time of detection and move the detection elements to be used on a one-by-one basis in the same direction for each time of detection, in a case where four times of detections are performed for acquiring detection signals for one acoustic image.

16. The photoacoustic image generation apparatus according to claim 4,
wherein the processor is further configured to set one detection element for continuous use and three detection elements for continuous suspension alternately and continuously in an order of arrangement of the detection elements of the detection element array for the detection elements to be used for one time of detection and move the detection elements to be used on a one-by-one basis in the same direction for each time of detection, in a case where four times of detections are performed for acquiring detection signals for one acoustic image.

17. The photoacoustic image generation apparatus according to claim 1,
wherein the processor is further configured to set two detection elements for continuous use and two detection elements for continuous suspension alternately and continuously in an order of arrangement of the detection elements of the detection element array for the detection elements to be used for one time of detection and move the detection elements to be used on a two-by-two basis in the same direction for each time of detection, in a case where two times of detections are performed for acquiring detection signals for one acoustic image.

18. The photoacoustic image generation apparatus according to claim 2,
wherein the processor is further configured to set two detection elements for continuous use and two detection elements for continuous suspension alternately and continuously in an order of arrangement of the detection elements of the detection element array for the detection elements to be used for one time of detection and move the detection elements to be used on a two-by-two basis in the same direction for each time of detection, in a case where two times of detections are performed for acquiring detection signals for one acoustic image.

19. The photoacoustic image generation apparatus according to claim 3,
wherein the processor is further configured to set two detection elements for continuous use and two detection elements for continuous suspension alternately and continuously in an order of arrangement of the detection elements of the detection element array for the detection elements to be used for one time of detection and move the detection elements to be used on a two-by-two basis in the same direction for each time of detection, in a case where two times of detections are performed for acquiring detection signals for one acoustic image.

20. The photoacoustic image generation apparatus according to claim 4,
wherein the processor is further configured to set two detection elements for continuous use and two detection elements for continuous suspension alternately and continuously in an order of arrangement of the detection elements of the detection element array for the detection elements to be used for one time of detection and move the detection elements to be used on a two-by-two basis in the same direction for each time of detection, in a case where two times of detections are performed for acquiring detection signals for one acoustic image.
